(19) United States
(12) Patent Application Publication
(10) Pub. No.: US 2018/0125627 A1
(43) Pub. Date: May 10, 2018

(54) TEETH WHITENING SYSTEM APPARATUS AND METHOD

(71) Applicant: Anthony D. Mounce, Kenner, LA (US)

(72) Inventor: Anthony D. Mounce, Kenner, LA (US)

(21) Appl. No.: 15/346,881

(22) Filed: Nov. 9, 2016

(51) Int. Cl. A61C 19/06; A61N 5/06; A61K 8/22; A61K 8/19

(52) U.S. Cl. CPC A61C 19/066; A61N 5/062; A61K 8/22; A61N 2005/0626; A61N 2005/0606; A61N 2005/0652; A61K 8/19

(57) ABSTRACT

A teeth whitening system with an improved tray and manner of application is provided. The teeth whitening tray of the present system comprises a U-shaped tray configured to receive a set of upper and lower dental ridges, or teeth, of a user. When in use, the user inserts the tray into his or her mouth in the appropriate manner, with the upper and lower dental ridges of the user being coated with the appropriate whitening agent. A plurality of LED lights are activated along the interior of the teeth whitening tray. The LED activation in turn activates the whitening agent and effectively whitens the user's teeth. A plurality of ridges is provided on the teeth whitening tray to ensure security of the teeth whitening agent during use, and upper and lower lip platforms are provided on the exterior of the teeth whitening tray to ensure the user's lips and other sensitive areas of the mouth are protected from the whitening agent during use.

### BACKGROUND OF THE INVENTION

The present invention provides a teeth whitening system with an improved tray and manner of application. The teeth whitening tray of the present system comprises a U-shaped tray configured to receive a set of upper and lower dental ridges, or teeth, of a user. When in use, the user inserts the tray into his or her mouth in the appropriate manner, with the upper and lower dental ridges of the user being coated with the appropriate whitening agent. A plurality of LED lights are activated along the interior of the teeth whitening tray. The LED activation in turn activates the whitening agent coated on the user's teeth, and effectively whitens the user's teeth. A plurality of ridges is provided on the teeth whitening tray to ensure security of the teeth whitening agent during use, and upper and lower lip platforms are provided on the exterior of the teeth whitening tray to ensure the user's lips and other sensitive areas of the mouth are protected from the whitening agent during use.

The teeth whitening system of the present invention is aimed at solving problems encountered from the use of complicated or relatively ineffective teeth whitening systems. People often employ such teeth whitening systems when their teeth become discolored due to habits that affect the whiteness of their teeth, such as drinking coffee or smoking cigarettes. In the past, teeth whitening systems were elaborate and expensive, and were generally only able to be applied by a dental professional. These systems may have required the dental professional to “paint” on a gel that may whiten the discolored teeth, or required the use of LED-lighted equipment that reacts with certain gels to create a whitening effect on the discolored teeth. In recent years, “at-home” teeth whitening kits have become available, whereby users may apply certain whitening gels to the teeth or employ particular whitening toothpastes that may utilize certain chemicals that whiten the teeth. However, the more simplistic at-home techniques are not as effective as the professional LED-powered equipment, and the more elaborate dental professional systems may be prohibitively expensive or inconvenient for the user.

This invention provides for a simple, user-managed system that nonetheless provides the effectiveness of the LED-powered dental professional equipment. The invention provides for an LED-powered whitening tray, which is formulated in the shape of a mouthpiece so that it may fit in the user's mouth and come in contact with the user's teeth. The user would apply the appropriate gel to the inner hollow of the tray, such that placement of the tray in the user's mouth would create contact between the whitening agent and the user's teeth. The tray is electrically-powered, and features a cord attached to the mouthpiece that may then be plugged into the appropriate power source. Once plugged in, the sixteen (16) blue LED lights lining the mouthpiece are activated, and the LED lighting shines on the gel in a manner that stimulates the active chemicals of the gel and effectively whitens the teeth.

As outlined below, various patents outline a teeth-whitening system relying on LED lighting to chemically whiten the teeth of the user, or a handheld whitening tray device. However, none of the prior art provides for a structure that prevents the whitening agent from contacting the user's outer mouth during application. Further, the prior art does not provide for elements of the teeth whitening tray that more effectively whiten the teeth of the user and make the system easier and more intuitive to use, as outlined in more detail below.

U.S. Pat. No. 8,940,033 issued to Joe Dwyer et al. on Jan. 27, 2015 for a “Method and Apparatus for Whitening Teeth.” The Dwyer apparatus comprises a mouth tray including a horseshoe-shaped trough sized to accommodate a dental ridge. A planar projection extends from the outer side of the trough so that its far end is external of the mouth. A light box containing a plurality of LEDs couples with the external end of the projection and illuminates the projection end. The mouth tray and projection are formed of a light-transmissive material to irradiate a peroxide gel in the trough to whiten the user's teeth.

U.S. Publication No. 2006/0019214, published Jan. 26, 2006 by Eric S. Lawrence et al. on a “Compact Tooth Whitening Device,” discloses a device that includes an electromagnetic radiation source and an applicator that is removably attached. A tooth whitening agent may be placed in the applicator and applied to the user's teeth. The electromagnetic radiation source is activated to impart electromagnetic radiation to the surface of the user's teeth, coated with the tooth whitening agent, thereby causing a chemical reaction with the whitening agent. The effect of this activation is to enhance and/or expedite the tooth whitening effect of the application. In one embodiment of the Lawrence invention, the tooth whitening agent is selected based upon the wavelength of electromagnetic radiation emitted from the electromagnetic radiation emitter. In another aspect of the invention, a “try me package” is provided to allow consumers to sample the invention on a treated portion of the product packaging.

U.S. Publication No. 2015/0064645 was published on Mar. 5, 2015 by Jennifer Jablow et al. on a “Photocatalytic Teeth Whitening” concept. The disclosed system relates to devices, methods, and kits for accelerating the rate of whitening teeth using an oxidizing agent, a photocatalytic agent, and a portable lightsource. The lightsource comprises a flashing blue light illuminator coupled to a removable mouthpiece lightguide that precisely directs the light to the teeth. The teeth are separately preconditioned with a bleaching material and a catalyst. The method disclosed by the publication comprises (1) preconditioning the tooth surface with one or more photocatalytic agents and one or more oxidizing agents, and (2) irradiating the tooth surface with light from an LED lightsource. In preferred embodiments, the photocatalytic agent may be zinc oxide, and the oxidizing agent may be hydrogen peroxide.

U.S. Pat. No. 7,331,784 issued Feb. 19, 2008 to Shuhei Suzuki on a “Mouthpiece.” The mouthpiece disclosed in the Suzuki invention includes a light irradiation section for irradiating the front surfaces of teeth with light having a predetermined wavelength, and where the light irradiation section has a blue light emitting diode or an ultraviolet light emitting diode. The mouthpiece is to be attached to the teeth, and in use, the front teeth are irradiated with a blue light or ultraviolet light emitted by the light emitting diode, thereby performing teeth whitening processing.

U.S. Publication No. 2006/0127837, published Jun. 15, 2006 by Bao Luu Nguyen et al. for “Dental Bleaching Using Regenerative Ionophoresis,” discloses a technique for dental bleaching. A tray covers the teeth of a user and has a reservoir. A conductor is placed inside the tray, which is covered by a bleaching agent when the bleaching agent fills the reservoir. The conductor is electrically connected to a negative terminal of a power source to cause regeneration of free radicals decomposed from the bleaching agent when power is applied. The tray may comprise a plurality of pockets that fit the teeth, the pockets forming the reservoir, and a groove formed along the pockets to enclose the conductor. The apparatus may further comprise an attachment electrically connected to a positive terminal of a power source and attached to the user.

U.S. Publication No. 2007/0184404 was published Aug. 9, 2007 by Bernd J. Johnki on a “Device for Cleaning And/Or the Care of Teeth And/Or Gums.” The publication discloses a device for cleaning and/or care of teeth and/or gums, the device comprising a molded, spoon-shaped mouthpiece for an upper jaw and/or a lower jaw, provided with at least one channel ending in at least one nozzle for applying liquid to the teeth and/or gums. The aim of the invention is to improve one such device for cleaning and/or care of teeth and/or gums, both in terms of the handling thereof and the obtainable cleaning and/or care effect on the teeth and/or gums, in such a way that effective and long-lasting cleaning and/or care of teeth and/or gums can be carried out in a more simple and comfortable manner, especially for people in need of care and confined to bed. To this end, the mouthpiece also comprises at least one channel ending in at least one opening, through which liquid applied to the teeth and/or gums can be essentially sucked out.

U.S. Pat. No. 8,029,278, issued on Oct. 4, 2011 to Jonathan B. Levine for an “Intra-Oral Whitening Device,” comprises a device that has a mouthpiece in which is embedded a flexible circuit board and arrays of spaced-apart lamps. The mouthpiece has a curvature. The lamps may be light-emitting diodes that generate electromagnetic radiation, preferably in the white and blue light spectrum and the infrared and ultraviolet light spectrum. The arrays are positioned to expose the facial and lingual sides of the teeth for whitening when the mouthpiece is positioned to fit upper and lower rows of teeth to be whitened within accommodating recesses. The flexible circuit board is flexed to exhibit a curvature that follows a curvature of the mouthpiece.

U.S. Publication No. 2013/0052613 was published Feb. 28, 2013 by Ramswamy Rangraj Chetiar et al. on a “Mouth Tray for Teeth Whitening.” The mouth tray includes a horseshoe-shaped main body made up preferably of a flexible material and anatomically contoured to fit exactly to the upper and lower sets of adult human teeth. The surface of the mouth tray has a plurality of elevated crisscross patterns to keep the whitening agent in place throughout the procedure. Both the ends of the mouth tray have elevated horizontal ridges with reduced elevation towards the interior portion of the mouth tray, which prevents the seeping and falling of the whitening agent during the process. A predetermined quantity of whitening gel may be prefilled on the mouth tray. The user just needs to attach the prefilled mouth tray to a suitable lighting device and place the mouth tray inside the mouth.

There is accordingly a need for a teeth whitening system that provides for an LED-stimulated whitening agent that is nonetheless user-friendly and able to be managed by a single user during application.

### SUMMARY OF THE INVENTION

The present invention provides a teeth whitening system that includes a teeth whitening tray designed for a single user to apply the whitening agent to the teeth, insert the teeth whitening tray appropriately, and activate the LED array of the whitening tray so as to whiten the teeth of the user. Because the teeth whitening tray provides for upper and lower lip platforms that prevent the whitening agent from contacting the user's lips or other sensitive areas of the mouth, the user may independently manage the application of the teeth whitening system without external assistance. The tray also provides for additional features that allow the tray to stay in the appropriate position during application, such that the user does not require external assistance, such as ridges lining the interior of the teeth whitening tray, acting as a grip for the teeth whitening agent, and a teeth platform on the interior of the teeth whitening tray, acting as a stabilizer for the user when biting down on the tray in the appropriate manner.

The present invention solves existing problems in the teeth whitening field, namely providing a user-managed system that nonetheless uses the power of an LED-activated teeth whitening agent to effectively whiten the user's teeth.

### BRIEF DESCRIPTION OF DRAWINGS

Reference will now be made to the drawings, wherein like parts are designated by like numerals, and wherein:

FIG. 1 is a perspective view of the teeth whitening system according to an embodiment of the present invention;

FIG. 2 is a front view of the teeth whitening tray shown in FIG. 1;

FIG. 3 is a back view of the teeth whitening tray shown in FIG. 1;

FIG. 4 is a top view of the teeth whitening system shown in the embodiment of FIG. 1;

FIG. 5 is a bottom view of the teeth whitening system shown in the embodiment of FIG. 1;

FIG. 6 is a right-side view of the teeth whitening system shown in the embodiment of FIG. 1; and

FIG. 7 is a left-side view of the teeth whitening system shown in the embodiment of FIG. 1, opposite the right-side view of FIG. 6.

### DETAILED DESCRIPTION OF THE DRAWINGS

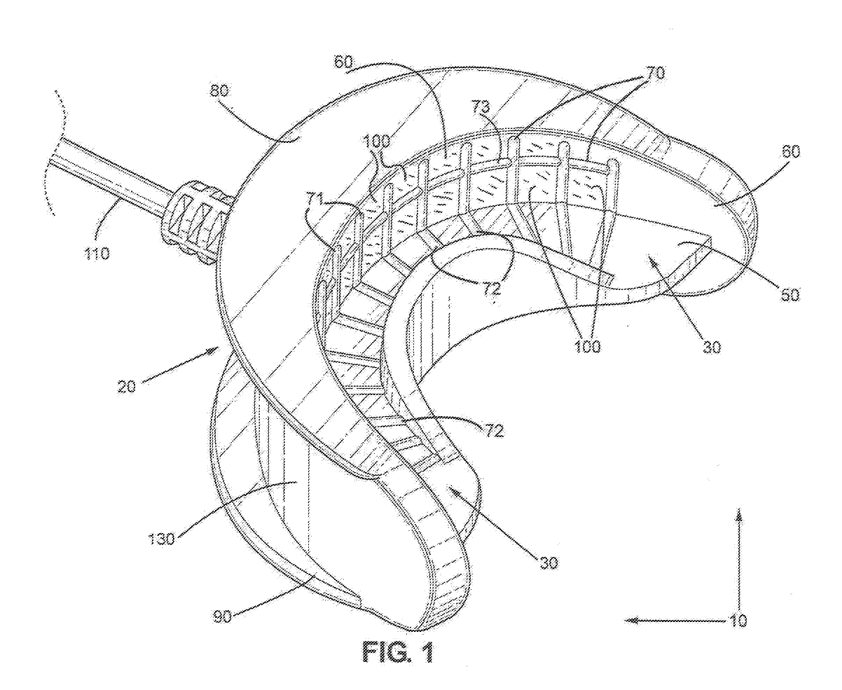

Referring to FIG. 1, an embodiment of the teeth whitening system 10 comprises a symmetrical U-shaped teeth whitening tray 20. In preferred embodiments of the teeth whitening system 10, the teeth whitening tray 20 is manufactured with plastic. However, the teeth whitening tray 20 may be made of any material that is transparent to the blue light emitted through the teeth whitening tray 20, as described in more detail herein.

The teeth whitening tray 20 features an upper trough 30, as well as a lower trough 40, depicted in FIGS. 1, 4, and 5. As depicted in FIG. 1, the troughs 30, 40 are formed by the boundaries created by a teeth platform 50 and vertical platform 60 of the teeth whitening tray 20. For example, upper trough is created by a bottom border of the teeth platform 50 and a longitudinal, or vertical, border of the vertical platform 60. Teeth platform 50 creates the separation between upper trough 30 and lower trough 40, such that lower trough 40 has a top border of the teeth platform 50 and a longitudinal, or vertical, border of vertical platform 60.

As shown in FIG. 1, teeth platform 50 is configured as a flat platform meant to be bitten down upon by the user during use of the teeth whitening system 10. Accordingly, as the user bites down on teeth platform 50 of the teeth whitening tray 20, the teeth of the user are aligned vertically against the vertical platform 60 of the teeth whitening tray 20, with the upper dental ridge of the user placed in the upper trough 30, and the bottom dental ridge of the user placed in the lower trough 40. The teeth platform 50 thus secures the teeth whitening tray in the appropriate position while the teeth whitening system is employed, and allows for the teeth whitening system to be used without the user manually holding the teeth whitening tray 20 in place with his or her hands during use. The provision of the teeth platform 50 further ensures that both the upper trough 30 and lower trough 40 may be appropriately accessed and utilized during use of the system. It may be understood that by providing for simultaneous use of the upper trough 30 and lower trough 40, the user may utilize the system one time, rather than two separate times, during whitening of the upper and lower dental ridges.

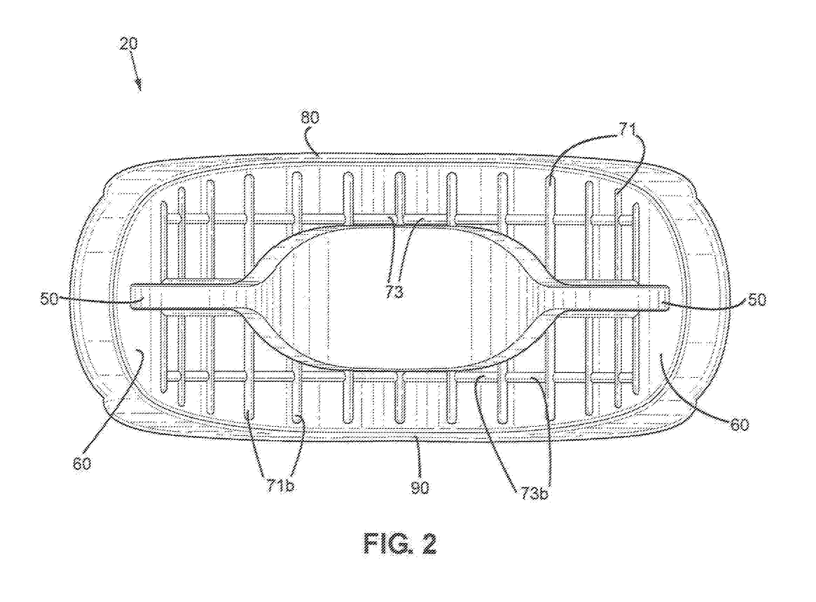

As depicted in FIG. 1, the teeth whitening tray 20 provides for ridges 70 along the interior of the teeth whitening tray 20. The ridges 70 comprise vertical ridges 71, horizontal ridges 72, and cross-ridges 73. As shown in FIG. 1, vertical ridges 71 are configured in even and symmetrical vertical lines along the vertical platform 60. Each vertical ridge 71 has a corresponding horizontal ridge 72, which extends along the teeth platform 50. Additionally, cross-ridges 73 are provided in between the spaces of vertical ridges 71. As shown in FIG. 1, cross-ridges 73 are lined in a horizontal manner relative to vertical ridges 71, and extend fully between each set of adjacent vertical ridges 71. As depicted in FIG. 2, a symmetrical second set of the vertical ridges 71B, corresponding horizontal ridges 72B, and cross-ridges 73B are provided in the bottom half of the teeth whitening tray 20 as separated by teeth platform 50.

As may be understood by those in the art, ridges 70 allow for further stability of the teeth whitening tray 20 when placed in the mouth of the user during use. Additionally, ridges 70 allow for increased stability of the teeth whitening gel when it is applied to the teeth whitening tray 20, as discussed in more detail below. Ridges 70 are provided in a manner to effectuate this stability, and are not meant to be limited to a certain number of vertical ridges 71, horizontal ridges 72, and cross-ridges 73, although preferred embodiments of the teeth whitening tray 20 may feature between four and twelve sets of vertical ridges 71, horizontal ridges 72, and cross-ridges 73, as depicted in the preferred embodiment of FIG. 1.

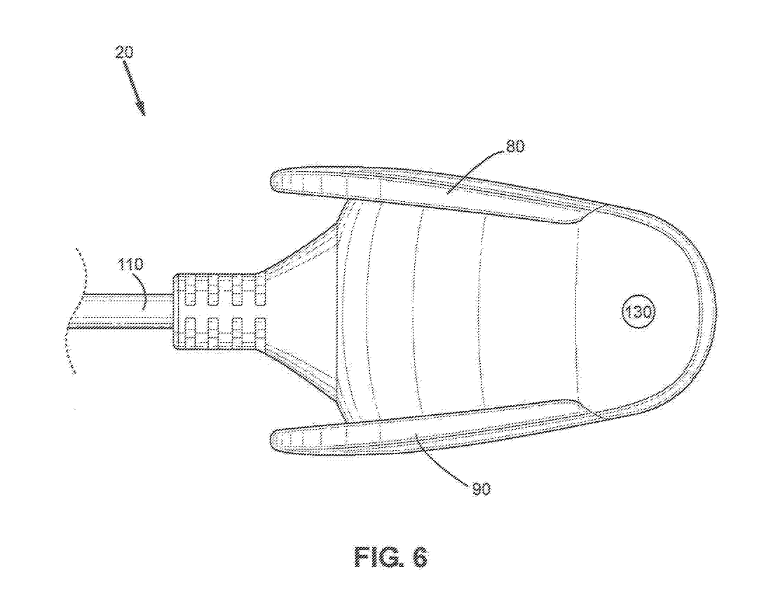

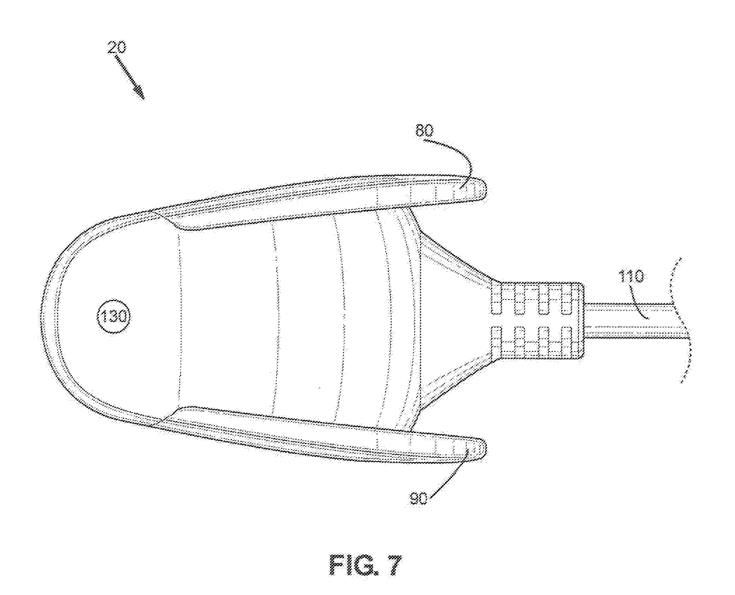

The teeth whitening tray 20 of the present invention further features additional platforms 80, 90 at the top and bottom of the teeth whitening tray 20, as shown in FIG. 1 and in perspective of FIG. 6 and FIG. 7. Exterior lip platforms 80, 90 are structured at slight upward and downward angles, respectively, extending outward from the top and bottom of exterior surface 130 of the vertical platform 60. Upper lip platform 80 and lower lip platform 90 are configured in a manner that when the teeth whitening platform 20 is inserted into the mouth of the user and the user bites down on teeth platform 50, the lips of the user rest above and below upper lip platform 80 and bottom lip platform 90, respectively. The lips of the user are therefore prevented from contacting the teeth whitening agent when it is applied to the teeth whitening tray 20 and subsequently positioned inside the mouth of the user. Avoidance of such contact is desirable during use of the teeth whitening system 10, as the teeth whitening agent may cause pain or otherwise create discomfort to the user if the lips or mouth interior of the user comes into contact with the teeth whitening agent. Provision of the top platform 80 and bottom platform 90 therefore serves not only as a resting position for the user's lips during use, but also provides crucial protection to the user's lips and other sensitive areas of the mouth during use.

As shown in FIG. 1, the teeth whitening tray 20 of the present invention features an embedded light source for activation of the teeth whitening system. An array of light-emitting diodes (“LED” array) 100 is provided along the curvature of the teeth whitening tray 20, as depicted in FIG. 1. The LED array 100 is able to emit blue light at a wavelength of between approximately 455 to 492 nanometers on the electromagnetic spectrum, and produces visible electromagnetic radiation, or light, from the LED array 100 when powered by an appropriate source. In preferred embodiments, the LED array 100 may feature sixteen individual LED lights on the tray, with eight individual LED lights along the top half of the teeth whitening system and the remaining eight individual LED lights along the bottom half of the teeth whitening system, which provides for even whitening when activated.

As shown in FIG. 1, the LED array 100 may be powered by USB cord 110, or other appropriate power source connection. A preferred embodiment of the teeth whitening system 10 may feature such a USB cord 110, wherein one end of the USB cord 110 is permanently affixed to the teeth whitening tray 20 at connection point 120 on the exterior surface 130 of vertical platform 60. The opposite end of the USB cord 110 may incorporate a USB male-connector that plugs into a corresponding USB port able to provide sufficient power to the teeth whitening tray 20.

It may be appreciated that because the LED array 100 does not require a significant source of power to activate, the USB cord 110 may be configured to plug into and appropriate power from almost any electronic device configured to receive a USB male-connector and function as a power source. For example, and without limitation, the teeth whitening system may be powered by a smartphone, such as an iPhone, but plugging the opposite end of the USB cord 110 into the USB port of the smartphone.

In use, the teeth whitening tray 20 of the present invention, and its LED array 100, is used in conjunction with a tooth whitening agent. The tooth whitening agent may preferably be a syringe-filled mixture of hydrogen peroxide, carbamide peroxide, sodium perborate, and sodium bicarbonate. In preferred embodiments of the present invention, the tooth whitening agent may be comprised of a hydrogen peroxide and carbamide peroxide base gel. In other preferred embodiments of the present invention, the tooth whitening agent may be comprised of sodium bicarbonate and perborate, which would exclude the use of peroxides in the base formula.

The user would apply this tooth whitening agent in a manner such that a gel coating is formed on the user's teeth, such as applying the agent directly to the teeth via a user-held instrument. Alternatively, the tooth whitening agent may be applied directly along the curvature of the vertical platform 60 of the teeth whitening tray 20. After application of the teeth whitening agent, either directly on the user's teeth or on the teeth whitening tray 20, the user would insert the teeth whitening tray 20 into his or her mouth and bite down on the teeth platform 50. By inserting the teeth whitening tray 20 into the mouth of the user and biting down on the teeth platform 50 in such a manner, the teeth whitening agent forms a gel coating upon the user's upper and lower dental ridges.

After proper insertion of the tray, the LED array 100 may thereafter be activated. It may be appreciated that although a preferred embodiment may feature a smartphone as the power source of the LED array 100, there may be embodiments in which the LED array 100 is powered by other appropriate power sources, such as Bluetooth technology, an electrical socket, or so forth. Upon provision of a power source to the teeth whitening tray 20, the LED array 100 is activated, and provides irradiation aimed at the user's upper and lower dental ridges. As a result of the interaction between the activated LED array 100 and the teeth whitening agent coated on the user's teeth, a photocatalyst within the teeth whitening agent is triggered that accelerates the decomposition of the whitening agent into its reactive components. This activation in turn increases the concentrations of the oxidizing agents in the interprismatic spaces within the enamel, resulting in the whitening of the user's teeth.

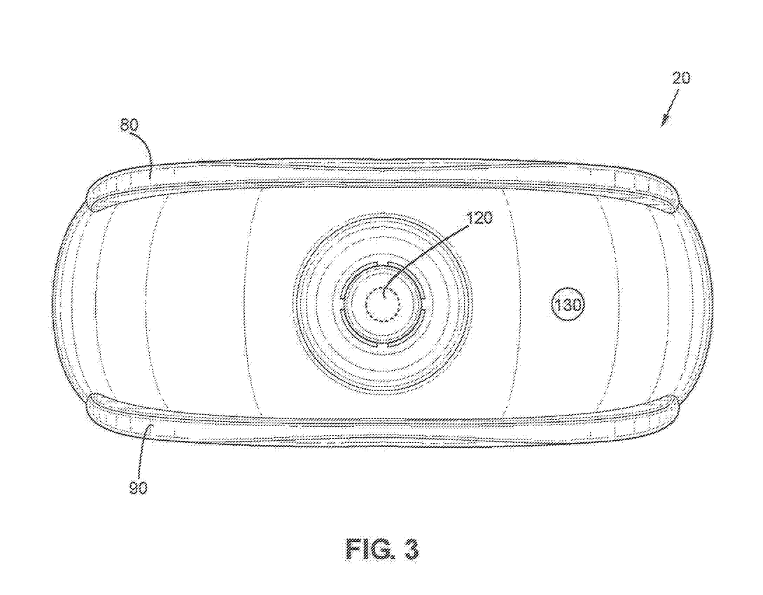

Referring to FIG. 2 and FIG. 3, respectively, a front and a back view of the teeth whitening tray 20 of the preferred embodiment of the invention are shown.

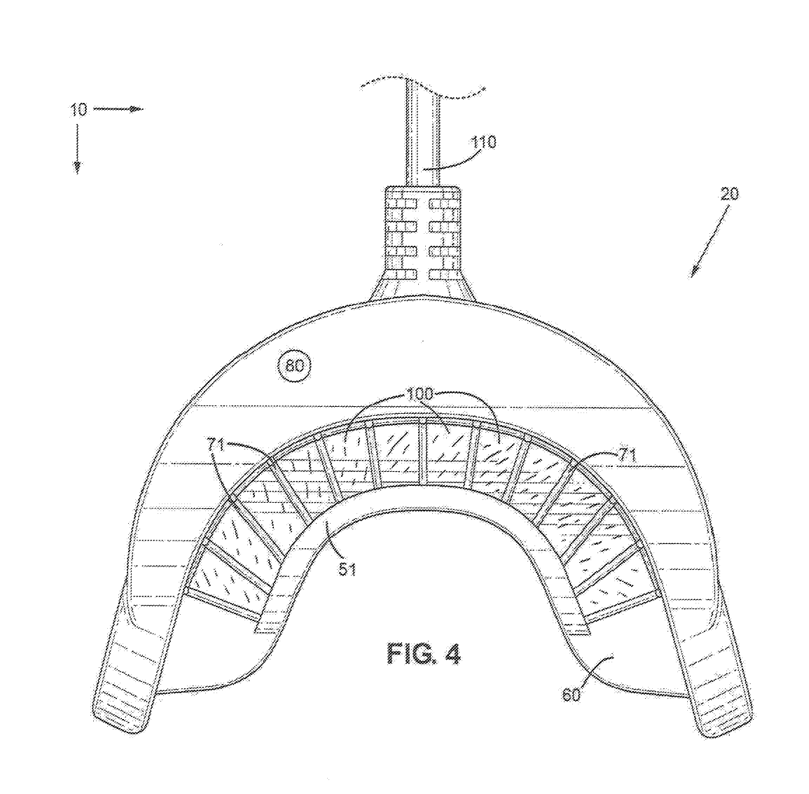

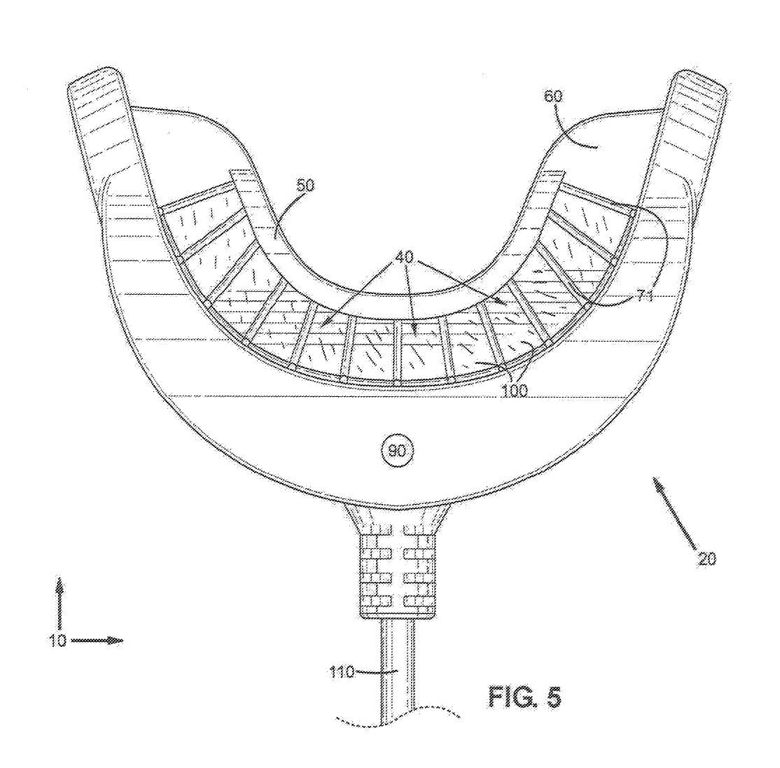

Referring to FIG. 4 and FIG. 5, respectively, a top and a bottom view of the teeth whitening tray 20 of the present invention are shown.

Referring to FIG. 6 and FIG. 7, respectively, a right-side and a left-side view of the teeth whitening tray 20 of the present invention are shown.

Many changes and modifications can be made in the present invention without departing from the spirit thereof. I therefore pray that my rights to the present invention be limited only by the scope of the appended claims.

I claim:

1. A teeth whitening system for whitening the teeth of a user, comprising:
(i) a teeth whitening tray structured in a symmetrical U-shape featuring:
(a) a vertical platform structured in said symmetrical U-shape and having an interior surface and an exterior surface;
(b) a horizontal teeth platform extending outwardly from said vertical platform at a point approximately halfway between the top and bottom of said vertical platform, creating a top vertical platform and a bottom vertical platform;
(c) a plurality of ridges, wherein said plurality of ridges comprise a set of vertical ridges arranged in vertical lines along said U-shaped vertical platform;
(d) an upper lip platform extending outward from said top of said vertical platform;
(e) a bottom lip platform extending outward from said bottom of said vertical platform; and
(f) a plurality of light-emitting diodes featured along said vertical platform;
wherein said vertical platform and said horizontal teeth platform form an upper trough and a lower trough on the interior of said teeth whitening tray; and
wherein said upper trough and said lower trough are configured to receive an upper dental ridge and a lower dental ridge of a user;
(ii) a power source, wherein said power source is operatively connected to said teeth whitening tray; and
(iii) a whitening agent;
wherein said power source provides power to said plurality of light-emitting diodes; and
wherein said plurality of light-emitting diodes activates said whitening agent when said power source provides power to said plurality of light-emitting diodes.

2. The teeth whitening system of claim 1, wherein said plurality of ridges further comprises a set of horizontal ridges arranged in horizontal lines along said horizontal teeth platform, and wherein said set of horizontal ridges are spaced in a manner to mirror said set of vertical ridges arranged in vertical lines along said U-shaped vertical platform.

3. The teeth whitening system of claim 1, wherein said plurality of ridges further comprises a set of horizontal ridges arranged in horizontal lines along said horizontal teeth platform, wherein said set of horizontal ridges are spaced in a manner to mirror the set of vertical ridges arranged in vertical lines along said U-shaped vertical platform, and a set of cross-ridges arranged in a single horizontal line along said U-shaped vertical platform between each individual ridge of said set of vertical ridges.

4. The teeth whitening system of claim 1, wherein said power source is operatively connected to said teeth whitening tray by a USB cord, and wherein said USB cord is connected to said teeth whitening tray at a connection point on said exterior surface of said vertical platform of said teeth whitening tray.

5. The teeth whitening system of claim 4, wherein said power source is a mobile device.

6. The teeth whitening system of claim 1, wherein said power source is operatively connected to said teeth whitening tray by Bluetooth technology.

7. The teeth whitening system of claim 1, wherein said whitening agent comprises a combination of hydrogen peroxide and carbamide peroxide.

8. The teeth whitening system of claim 1, wherein said whitening agent comprises a combination of sodium bicarbonate and perborate.

9. The teeth whitening system of claim 1, wherein said plurality of light-emitting diodes comprises eight light-emitting diodes featured along said top vertical platform and eight light-emitting diodes featured along said bottom vertical platform.

10. The teeth whitening system of claim 1, wherein said upper lip platform extends outward from said top of said vertical platform at an upward angle, and wherein said bottom lip platform extends outward from said bottom of said vertical platform at a downward angle.

11. A method for whitening teeth of a user, comprising:
(i) providing a teeth whitening system comprising:
(a) a teeth whitening tray structured in a symmetrical U-shaped featuring:
(1) a vertical platform structured in said symmetrical U-shape and having an interior surface and an exterior surface;
(2) a horizontal teeth platform extending outwardly from said vertical platform at a point approximately halfway between the top and bottom of said vertical platform, creating a top vertical platform and a bottom vertical platform;
(3) a plurality of ridges, wherein said plurality of ridges comprise a set of vertical ridges arranged in vertical lines along said U-shaped vertical platform;
(4) an upper lip platform extending outward from said top of said vertical platform;
(5) a bottom lip platform extending outward from said bottom of said vertical platform; and
(6) a plurality of light-emitting diodes featured along said vertical platform;
wherein said vertical platform and said horizontal teeth platform form an upper trough and a lower trough on the interior of said teeth whitening tray; and
wherein said upper trough and said lower trough are configured to receive an upper dental ridge and a lower dental ridge of a user;
(b) a power source, wherein said power source is operatively connected to said teeth whitening tray; and
(c) a whitening agent;
(ii) providing power to said plurality of light-emitting diodes from said power source; and
(iii) activating said whitening agent with said plurality of light-emitting diodes when said power source provides power to said plurality of light-emitting diodes.

12. The method for whitening teeth of a user of claim 11, wherein said plurality of ridges further comprises a set of horizontal ridges arranged in horizontal lines along said horizontal teeth platform, and wherein said set of horizontal ridges are spaced in a manner to mirror the set of vertical ridges arranged in vertical lines along said U-shaped vertical platform.

13. The method for whitening teeth of a user of claim 11, wherein said plurality of ridges further comprises a set of horizontal ridges arranged in horizontal lines along said horizontal teeth platform, wherein said set of horizontal ridges are spaced in a manner to mirror the set of vertical ridges arranged in vertical lines along said U-shaped vertical platform, and a set of cross-ridges arranged in a single horizontal line along said U-shaped vertical platform between each individual ridge of said set of vertical ridges.

14. The method for whitening teeth of a user of claim 11, wherein said power source is operatively connected to said teeth whitening tray by a USB cord, and wherein said USB cord is connected to said teeth whitening tray at a connection point on said exterior surface of said vertical platform of said teeth whitening tray.

15. The method for whitening teeth of a user of claim 14, wherein said power source is a mobile device.

16. The method for whitening teeth of a user of claim 11, wherein said power source is operatively connected to said teeth whitening tray by Bluetooth technology.

17. The method for whitening teeth of a user of claim 11, wherein said whitening agent comprises a combination of hydrogen peroxide and carbamide peroxide.

18. The method for whitening teeth of a user of claim 11, wherein said whitening agent comprises a combination of sodium bicarbonate and perborate.

19. The method for whitening teeth of a user of claim 11, wherein said plurality of light-emitting diodes comprises eight light-emitting diodes featured along said top vertical platform and eight light-emitting diodes featured along said bottom vertical platform.

20. The method for whitening teeth of a user of claim 11, wherein said upper lip platform extends outward from said top of said vertical platform at an upward angle, and wherein said bottom lip platform extends outward from said bottom of said vertical platform at a downward angle.
